(19) United States
(12) Patent Application Publication
(10) Pub. No.: US 2018/0052272 A1
(43) Pub. Date: Feb. 22, 2018

(54) LIGHT SPREADING IN TEXTILES

(71) Applicant: PHILIPS LIGHTING HOLDING B.V., EINDHOVEN (NL)

(72) Inventors: HUGO JOHAN CORNELISSEN, EINDHOVEN (NL); JIANGHONG YU, EINDHOVEN (NL); GIOVANNI CENNINI, EINDHOVEN (NL); PHILIPPE BERTRAND DANIEL GUERMONPREZ, EINDHOVEN (NL); PIERRE JEAN BERNARD BOUVIER, EINDHOVEN (NL)

(21) Appl. No.: 15/558,558

(22) PCT Filed: Mar. 10, 2016
(86) PCT No.: PCT/EP2016/055161

§ 371 Date: Sep. 14, 2017

(30) Foreign Application Priority Data
Mar. 17, 2015 (EP) 15159356.3

(51) Int. Cl. F21V 8/00

(52) U.S. Cl. CPC G02B 6/0021; A61N 2005/063

(57) ABSTRACT

The present invention relates to a light emitting device comprising a light emitting element and a light guide arranged on a textile substrate and wherein one or more air gaps are present in the light guide at the interface with the textile substrate. The method also relates to a method of making the light emitting device and to various uses of the light emitting device.

### FIELD OF THE INVENTION

The present invention relates to a light emitting device comprising a light emitting element and a light guide arranged on a textile substrate. The method also relates to a method of making the light emitting device and to various uses of the light emitting device.

### BACKGROUND OF THE INVENTION

By integrating light sources, such as light emitting diodes (LEDs), into textiles and thereby creating light emitting electronic textiles, visual effects can be achieved. Efforts have also been made to form textile integrated display devices.

However, LEDs are point source emitters, so a textile with integrated LEDs tends to appear as bright dazzling spots and the appearance does not always create an appealing look. For other applications, such as for light therapy uses, it may be important that the light is emitted in a more diffuse and homogeneous manner.

Existing light emitting electronic textiles are often provided with a textile diffuser arranged on top of the light sources to achieve a more uniform output of light from the light emitting electronic textile. Many of these textile diffusers tend to absorb a substantial proportion of the light emitted by the light sources and/or to suffer from increasing absorption over time. There is also room for improving the mechanical properties of existing light emitting electronic textiles. WO 2010/122458 addresses these problems but still requires the presence of a flexible light spreading layer for distancing the light sources from the covering textile and for allowing light emitted by the light sources to spread before hitting the covering textile. WO 2013/046113 describes a heat recovery system for a light therapy device comprising a heat spreading section.

There is still a need for alternative and/or improved light emitting textiles, and methods of making thereof, wherein a uniform spread of light is achieved with reduced and/or minimal bright spots and at the same time a thin and flexible system is provided with good mechanical properties.

### SUMMARY OF THE INVENTION

It is an object of the present invention to overcome these problems, and, inter alia, to provide a light emitting textile device and a method of making the device. It would be advantageous to achieve good levels of light uniformity with minimal/decreased bright spots and a thin and flexible system.

According to a first aspect of the invention, these and other objects may be achieved by a light emitting device comprising:

at least one light emitting element arranged on a first surface of a textile substrate;

a first light guide arranged to coat the at least one light emitting element;

wherein the first light guide has a back surface facing the first surface of the textile substrate and a front surface facing away from the textile substrate;

wherein the back surface of the first light guide is in direct contact with the first surface of the textile substrate and forms an interface with the first surface of the textile substrate and wherein one or more air gaps may be present in the first light guide at the interface with the textile substrate;

wherein a mask is deposited on the front surface of the first light guide and over the at least one light emitting element.

The light guide may coat at least the surfaces of the at least one light emitting element which are not arranged on or in contact with the textile substrate. For those surfaces of the light emitting element which are not in contact with the textile substrate, there may be no air gap between the surfaces of the light emitting element and the first light guide. The at least one light emitting element may be at least partially embedded in the textile substrate. This may depend to some extent on the nature of the textile.

In a second aspect, the invention provides a method of forming a light emitting device in accordance with the first aspect of the invention. The method comprises:

arranging at least one light emitting element on a first surface of a textile substrate;

depositing a sacrificial material on said first surface of the textile substrate;

coating the at least one light emitting element, the first surface of the textile substrate and the sacrificial material with a first light guide;

removing the sacrificial material;

depositing a mask on the first light guide and over the at least one light emitting element.

The sacrificial material may be removed before or after the mask is deposited on the light guide. The light of the at least one light emitting element, e.g. one or more LEDs or a plurality of LEDs, is spread uniformly by using a transparent coating as a first light guide layer. In order to prevent the guided light from uncontrolled scattering off the textile and thus creating bright spots, the coating is detached from the textile so that only some of the back surface of the first light guide may be in direct contact with the first surface of the textile substrate. This is established using a sacrificial layer or layers. After the coating step, this sacrificial layer(s) is removed creating the required air gap(s).

The sacrificial material may have a pattern of holes or gaps to obtain bonding areas between the first light guide and the textile. The bonding areas provide mechanical contact and light extraction regions or features. The sacrificial material may be a textured surface or possess a particular shape in order to obtain a homogeneous or directional distribution of light (e.g. a “V”-cut).

The at least one light emitting element, the first surface of the textile substrate and the sacrificial material may be coated by applying the light guide in the form of a liquid, e.g. a liquid polymer, followed by curing. The liquid light guide may be applied using printing, stencilling or dispensing. Following coating of the at least one light emitting element, the first surface of the textile substrate and the sacrificial material, the first light guide may be cured. A further or second light guide may be deposited on the first light guide. The second light guide may be cured.

The mask may comprise scattering and/or reflective and/or absorbing structures or features. For example, the mask may comprise reflective white dots of ink. The mask may be heated or cured after it is deposited on the first light guide and over the at least one light emitting element. The mask may comprise a structure or structures which will absorb light which is reflected at steep angles and which will not be captured in the light emitting device and totally internally reflected therein. These structure(s) may comprise a light absorbing structure or structures. Said structure or structures may redirect light emitted from the at least one light emitting element and which is not totally internally reflected into the light guide or light guides. For example, the light absorbing structure or feature may comprise a black ring or rings. The black ring or rings may surround the rest of the mask, for example the black ring or rings may surround reflective white dots of ink. The light absorbing structure or structures may comprise an assembly of mirrors, for example an assembly of tilted mirrors. These arrangements serve to reduce the number of bright spots close to the light emitting element, e.g. LED or plurality of LEDs.

The guided light can be extracted uniformly over the surface by a pattern of light extraction features or by contact with the textile.

By the term “textile”, is herein meant a material or product that is wholly or partly made of textile fibers. The textile may, for example, be manufactured by means of weaving, braiding, knitting, crocheting, quilting or felting. In particular, a textile may be woven or non-woven. Non-woven textiles include, for example, felt, and foam. The textile substrate is sufficiently permeable that the sacrificial material can be removed through the textile substrate either by application of a solvent or heat.

By the term “at least one light emitting element arranged on a first surface of a textile substrate”, is herein meant the at least one light emitting element may be arranged on a surface of the textile or the at least one light emitting element may be embedded or partially embedded in the textile substrate.

The terms “transparent”, “opaque” and “transmissive” relate to the optical properties of particular components of the device relative to the wavelength of the light generated by the incorporated at least one light emitting element.

In further aspects, the invention provides numerous uses and applications of the light emitting devices made in accordance with the present invention. As such, and in a further aspect of the present invention there is provided an article or device comprising a light emitting device in accordance with the first aspect of the present invention. For example, the light emitting device may be used for lighting (e.g. in a luminaire), including in functional lighting, decorative lighting, lighting for logos, lighting in upholstery or in curtains, personal health care, in health care devices, for pain relief, for psoriasis and/or other skin treatment, for treating jaundice, e.g. jaundice baby blankets or sheets. The light emitting device may be comprised in a plaster, a blanket or a sheet.

An advantage of the light emitting device according to the present invention is that the amount of light scattering from the textile surface in an uncontrolled manner is reduced thus reducing the occurrence of so-called bright spots. The light which is guided in the light guide can be extracted uniformly over an output surface of the light emitter. This may be achieved by using a pattern of extraction features and/or by local contact with the surface of the textile. Light may be extracted through the front surface and/or the back surface of the light guide. The surface through which the light is emitted from the light emitting device may be referred to herein as an output surface.

It is noted that the invention relates to all possible combinations of features recited in the claims.

### BRIEF DESCRIPTION OF THE DRAWINGS

This and other aspects of the present invention will now be described in more detail, with reference to the appended drawings showing embodiment(s) of the invention.

FIG. 1 shows a flow chart depicting one example of preparing a light emitting device according to the present invention.

FIGS. 2a-d show cross sectional side views of the steps for preparing a light emitting device according to embodiments of the invention and FIG. 2e shows a plan view.

FIGS. 3a-3d show cross sectional side views of the steps for preparing a light emitting device according to embodiments of the invention.

FIGS. 4a-4d show cross sectional side views of the steps for preparing a light emitting device according to embodiments of the invention.

### DETAILED DESCRIPTION

The present invention will now be described more fully hereinafter with reference to the accompanying drawings, in which currently preferred embodiments of the invention are shown. This invention may, however, be embodied in many different forms and should not be construed as limited to the embodiments set forth herein; rather, these embodiments are provided for thoroughness and completeness, and fully convey the scope of the invention to the skilled person. Like reference numerals in the drawings refer to like elements throughout.

The present inventors have found that the appearance of bright light spots emitted from a light emitting device may be lessened by the presence of one or more air gaps at the interface of a light guide and a textile substrate.

The textile substrate may be made from any suitable textile. The main criteria is that the textile substrate should be sufficiently permeable in order to allow the sacrificial material to be removed after the light guide has been applied to the surface of the textile substrate, the sacrificial material and the at least one light emitting element. The textile substrate is selected from a material or product that is wholly or partly made of textile fibers. The textile may, for example, be manufactured by means of weaving, braiding, knitting, crocheting, quilting or felting. In particular, a textile may be woven or non-woven. Non-woven textiles include, for example, felt, and foam. Specific examples of textiles for use in the present invention include one or more of the following: cotton, wool, polyester, nylon, woven polyester, e.g. woven polyester filaments. The textile substrate should also be suitable for enabling attachment of the at least one light emitting element, e.g. at least one LED or a plurality of LEDs. The at least one LED or plurality of LEDs may consist of or comprise side emitting LEDs.

The at least one light emitting element, e.g. at least one LED or plurality of LEDs, may be mounted on a printed circuit board. The printed circuit board may be attached to the textile. For example, the printed circuit board may be attached to the textile by embroidery. The printed circuit board may be a flexible printed circuit board. The at least one light emitting element, e.g. at least one LED or plurality of LEDs, may be soldered to the textile substrate, for example using low temperature solder. The at least one light emitting element, e.g. at least one LED or plurality of LEDs, may be attached, or soldered (e.g. using low temperature solder), or clamped to conductive filaments that are combined with, e.g. woven into, the fabric, or they may be (pre)-attached to a textile ribbon which comprises conductive wires.

The light guide may be applied to the surface of the textile on which the at least one light emitting element is arranged so that the light guide coats the surface of the textile substrate and the available surfaces of the at least one light emitting element and the sacrificial material. Once the light guide has been applied or deposited, the at least one light emitting element may be described as being encapsulated by the textile substrate on which it is arranged (optionally, via a further substrate, such as a printed circuit board) and the light guide. There is no air gap directly between the light emitting element and the coating of light guide. However, due to the nature of the fabric and the fact that it has to be sufficiently permeable to allow the sacrificial material to be removed this means that some air may be present at the interface of the at least one light emitting element and the surface of the textile substrate to which it may be attached.

The at least one light emitting element may be selected from at least one LED. The at least one LED may be selected from one or more side emitting LEDs. The at least one LED may be selected from one or more top emitting LEDs or a combination of side emitting LEDs and top emitting LEDs. The LED(s) is arranged so that light from the LED(s) is optically coupled or directed into the first light guide and is totally internally reflected through the light guide and across the interface formed by the light guide and the air gap. The LED(s), e.g. plurality of LEDs, may be arranged across the surface of the textile substrate forming a number of rows and columns. The LED(s) may be arranged towards the edge of the textile substrate. The LEDs may be arranged in a regular or an irregular pattern. Light from the at least one light emitting element is totally internally reflected in the light guide until it is disrupted and the light is redirected in a direction out of the light guide and out of the light emitting device. Broadly, this may be in a direction which is substantially normal to the first surface of the textile substrate. The totally internally reflected light may be disrupted when it is incident on light extraction regions. An extraction region is typically formed where the first light guide is in direct contact with the textile substrate. An extraction region may also be provided by a pattern of extraction features. The application of the extraction features may be achieved by printing, micromoulding, microstamping or microembossing methods. Suitable extraction features may be in the form of a patterned ink layer which may be reflecting. The extraction features may be reflective printed ink dots wherein each dot disturbs the total internal reflection of the guided light and causes the light to be scattered randomly and to escape from the light guide. The size and/or pitch of the dots may be varied to ensure uniform light scatter. The extraction features may be applied using a printing process, e.g. screen printing incorporates the use of a mesh screen with openings corresponding to the pattern required to be printed. Suitable inks may be solvent or UV cured. Other suitable extraction features include microstructured surfaces which comprise a plurality of three dimensional features which are proud of the surface and arranged on a suitable scale. The extraction features may be present on the first surface of the textile substrate and may be deposited before or after the sacrificial material is deposited.

The sacrificial material (or layer) is a material that may be readily dissolved in a solvent or may sublime on heating. This enables the sacrificial material to be easily removed after the first light guide has been applied without adversely affecting the light emitting device. The sacrificial material may be deposited by any one of a range of techniques. For example, the sacrificial material may be deposited by sputtering through a mask, frame printing, 3D printing, thermal transfer, gluing. Suitable materials for the sacrificial material may be selected from one or more of the following: polyvinyl alcohol, guar, beeswax.

The sacrificial material may be removed by bringing the sacrificial material into contact with a suitable solvent, for example an aqueous solvent or an organic solvent depending on the nature of the sacrificial material. For example, a suitable solvent for polyvinyl alcohol is water. The sacrificial material may also be selected from a material that sublimes. For example, the sacrificial material may sublime at a low temperature. Sublimation may be effected by use of an oven. Removal of the sacrificial material provides an air gap or air gaps. The air gap or air gaps may be referred to herein as an air layer or air layers. Where the air gap is formed, the first light guide may be described as being locally detached from the textile substrate or more generally as an area of local detachment. The guided light may be extracted uniformly over the surface by a pattern of extraction features or by contact with an area of local detachment.

The first light guide may be made from any of the materials which are known for use as light guides. Suitable materials are transparent or light transmissive. Transparent polymers are suitable materials. Suitable polymers are selected from silicone, polyurethane, polyvinylchloride (PVC). Examples of suitable silicones are 7070 MOMENTIVE LSR, LSR 7060, Dow corning Sylgard 184. The thickness of the first light guide may be about 1 mm to about 5 mm, for example, about 1 mm to about 2 mm. The refractive index of the first light guide may be about 1.4 to about 1.6, for example, about 1.42 to about 1.5. The first light guide may be arranged on the first surface of the textile substrate by coating or casting. Following coating with the first light guide, the first light guide may be cured.

A first mask is deposited on the light guide and over the at least one light emitting element. The first mask may be deposited on the front surface of the first light guide. The mask may be heated or cured after it is deposited on the first light guide and over the at least one light emitting element. The mask may be positioned to absorb and/or reflect light incident at an angle which is not totally internally reflected by light reflected by the light guide/air gap interface. The mask may be an ink deposited by a suitable method. Suitable methods include printing, for example screen printing. The mask may comprise or consist of a black and/or white ink. The ink may be deposited by screen printing.

The mask may comprise scattering structures and/or reflective structures and/or absorbing structures or features. The mask may comprise reflective white dots of ink. The mask may comprise a structure or structures which will absorb light which is reflected at steep angles and which will not be captured in the light emitting device and totally internally reflected therein. For example, the light absorbing structure or structures may comprise a black ring or rings. The black ring or rings may surround the rest of the mask, for example the black ring or rings may surround the reflective white dots of ink. This arrangement serves to reduce the number of bright spots close to the light emitting element, e.g. LED or plurality of LEDs. While the black ring or rings is typically circular in shape, shapes varying from a regular circular shape may also be used in embodiments of the invention. The ring(s) and/or mask may, for example, form a circular, elliptical, square or rectangular shape.

Optionally, a second mask may be applied at the interface between the light emitting element(s) and the textile substrate. The second mask may possess the same features as the first mask. The second mask is applied prior to the at least one light emitting element being arranged on the textile substrate.

A further or second light guide may be deposited on the first light guide. The second light guide may be made from the same material as the first light guide. Suitable materials are transparent or light transmissive. Transparent polymers are suitable materials. Suitable polymers are selected from silicone, polyurethane, polyvinylchloride (PVC). Examples of suitable silicones are 7070 MOMENTIVE LSR, LSR 7060, Dow corning Sylgard 184. The thickness of the further or second light guide may be about 1 mm to about 3 mm, for example, about 1 mm to about 2 mm. The refractive index of the second light guide may be about 1.4 to about 1.6, for example, about 1.4 to about 1.5. The second light guide may have the same refractive index as the first light guide or a refractive index which is greater than the first light guide. The further or second light guide layer may be arranged on the front surface of the first light guide by coating or casting. Following coating with the further light guide, the further light guide may be cured.

The effect of depositing the further or second light guide is to sandwich the first mask at the interface between the first and second light guides. This also allows for the light to be more evenly distributed over the output surface. The light guides may be combined using a standard lamination technique. Such a technique may require the use of a transparent adhesive which has a refractive index which is equal to or higher than both the first and second light guides. The light guides may be joined optically during manufacture. The method of combining the layers may comprise applying and curing a liquid polymer layer. Curing may be undertaken using one or more techniques including UV, thermal or two-part curing. The method may comprise stenciling, dispensing or printing the liquid polymer. Optically joined indicates that the light guides or layers are combined in such a way that optically these layers are effectively indistinguishable.

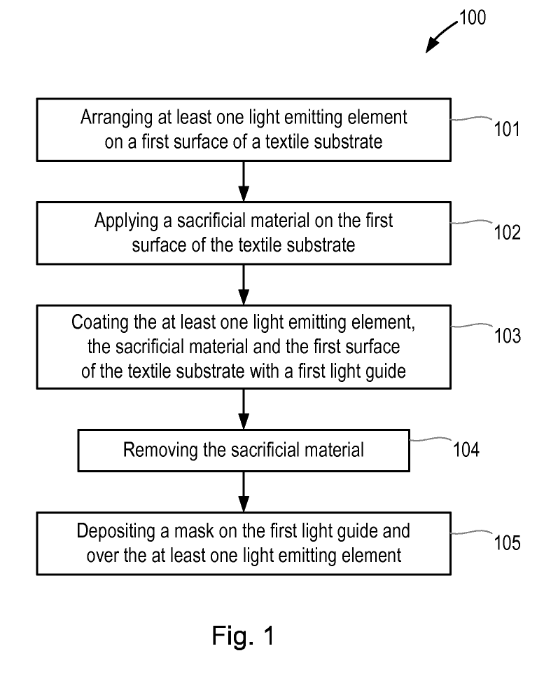

FIG. 1 shows a flow chart of a method (100) of preparing a light emitting device according to the present invention. In a first step (101), at least one light emitting element is arranged on a first surface of a textile substrate. The at least one light emitting element may be mounted on a flexible printed circuit board which may be attached to the textile substrate by embroidery. The at least one light emitting element may be embedded at least partially or completely within the first surface of the textile substrate. In a second step (102), a sacrificial material is applied to a part or parts of the first surface of the textile substrate. The sacrificial material may be applied using a suitable mask. In a third step (103), the at least one light emitting element, the first surface of the textile substrate and the sacrificial material are coated with a first light guide. In a fourth step (104) the sacrificial material is removed and in a fifth step (105) a mask is deposited on the first light guide and over the at least one light emitting element. In an optional sixth step (not shown) a further, (or second), light guide may be deposited on the first light guide, sandwiching the mask at the interface between the first and second guide layers. In a further optional step (not shown), a second mask may be applied to the substrate prior to the arrangement of the at least one light emitting element so that the second mask is sandwiched between the textile substrate and the at least one light emitting element.

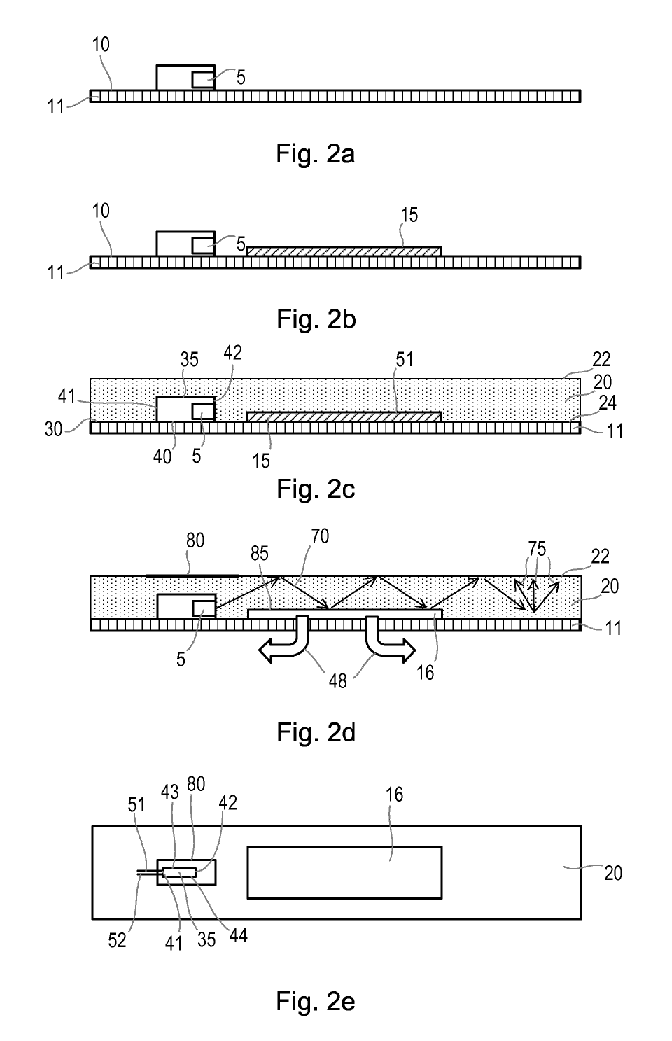

FIGS. 2a-2d show a number of cross sectional side views in connection with the construction of a light emitting device in accordance with the present invention. In FIG. 2a, a light emitting element (5), such as an LED is arranged on a first surface (10) of a textile substrate (11). In FIG. 2b, a sacrificial material (or layer) (15) is deposited on the first surface (10) of the textile substrate (11). In FIG. 2c, a light guide (20) is deposited on to the light emitting element (5), the sacrificial material (15) and the first surface (10) of the textile layer. The light guide has a front surface (22) and a back surface (24) and the back surface (24) is in contact with the first surface (10) of the textile substrate (11) to form an interface (30) therewith. The back surface of the light guide (24) is also in contact with the sacrificial material (15) to form an interface (51) therewith. The light emitting element (5) has a top surface (35) and a bottom surface (40) and a number of side surfaces (41, 42, 43, 44). The light guide (20) is shown coating the entirety of the top surface (35) and side surfaces (41-44) of the light emitting device. FIG. 2e shows a plan view of the arrangement shown in FIG. 2d. In FIG. 2e, the light guide (20) is shown in contact with the sides (41-44) and is in contact with the entirety of the sides (41-44) so that the light emitting element (5) is effectively encapsulated by the light guide (20) and the first surface of the textile substrate (11). In FIG. 2d, the arrows (48) indicate the sacrificial material (15) being removed to leave an air gap (16). The sacrificial material (15) may be removed by using a solvent to dissolve the sacrificial material (15) and provide an air gap (16). The sacrificial material (15) may be removed by exposing the sacrificial layer to heat. By exposing the sacrificial material to heat, the sacrificial material may sublime leaving an air gap (16). A light guide/air gap interface is indicated at (85). A mask (80) is deposited or placed on the front surface (22) of the light guide (20) so that at least some or all of the light emitting element (5) is masked. The mask (80) may be positioned to absorb and/or reflect light incident at an angle which is not totally internally reflected by light reflected by the light guide/air gap interface (85). The mask (80) may be an ink (e.g. black and/or white ink) deposited by a suitable method. Suitable methods include printing, for example screen printing. Light (70) is shown being emitted from the light emitting element (5) and being totally internally reflected through the light guide (20). The propagation of the light is disrupted when it is incident on the textile surface and is shown (75) being redirected in the direction of the front surface (22) of the light guide. This redirection of the totally internally reflected light may be referred to as disruption of the guided light. In FIG. 2e, the connector wires to the light emitting element (5) are indicated at (51, 52). In use, the area corresponding to the air gap (16) appears darker and more light is extracted starting at the edge of the air gap where the guide layer (20) is in contact with the first surface (10) of the textile substrate (11). Optionally, a second mask (not shown) may be applied at the interface between the light emitting element (5) and the textile substrate (11).

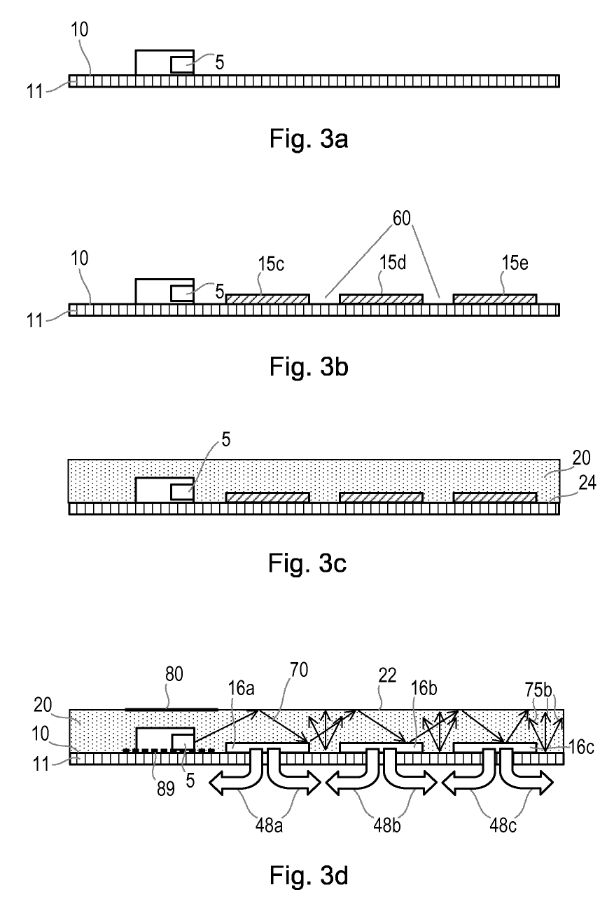

FIGS. 3a-3d show the same arrangement as in FIGS. 2a-2d apart from in FIG. 3b the sacrificial material (15), indicated as (15c, 15d, 15e), is present with a number of holes or gaps (60). Removal of the sacrificial material (or layers) (15c, 15d, 15e) is indicated at (48a, 48b, 48c) in FIG. 3d. The propagation of the light is disrupted where the first layer of light guide is in contact with the textile substrate and is shown (75b) being redirected in the direction of the front surface (22) of the light guide. FIG. 3d shows the optional presence of a second mask (89) at the interface between the light guide (20) and the textile substrate (11). The optional second mask (89) is deposited before the at least one light emitting element (5) is arranged on the surface of the textile substrate (10). The second mask (89) may be deposited using the same techniques and using the same materials as for the mask (80). The second mask (89) may also be present in the embodiments described and shown in FIGS. 1, 2a-2e and 4a-4d though it is not shown.

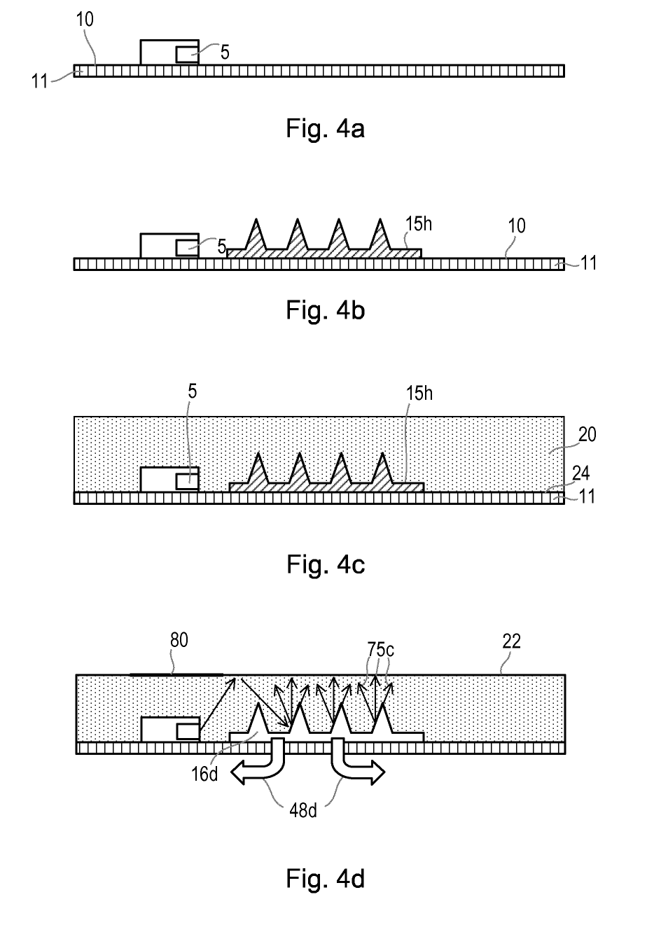

FIGS. 4a-4d show the same arrangement as in FIGS. 2a to 2d apart from a textured layer of sacrificial material (15h) is deposited on the first surface (10) of the textile substrate (11) in order to create an air gap which may be referred to as a textured air gap (16d). Removal of the sacrificial material (15h) is indicated at (48d) in FIG. 4d. The propagation of the light is disrupted where the light is incident on the textured air gap and is shown (75c) being redirected in the direction of the front surface (22) of the light guide. The front surface (22) may be referred to as an output surface.

### EXAMPLES

The inventors investigated the ability of a light emitting device in accordance with the present invention to remove, or extract, light from a light guide uniformly.

### Example 1: Preparation of a Light Emitting Device Comprising an LED, Water Soluble Sacrificial Material and a Black Ink Mask

A light emitting device in accordance with the present invention is made as follows. An LED is secured to the surface of a fabric of woven polyester filaments by soldering to conductive wires that are woven inside the fabric. A water-soluble sacrificial layer of polyvinylalcohol is deposited on the surface of a fabric. The sacrificial layer is deposited by sputtering through a mask. A transparent layer of silicone (LSR 7060, available from Momentive) is applied to the surface of the fabric, the sacrificial layer and the LED by coating and is cured. The sacrificial layer is removed by exposing it to water. The permeability of the fabric allows the sacrificial layer to be removed by passage through the fabric. A mask comprising black ink is deposited on the layer of cured silicone and positioned over the LED by screen printing and is cured.

### Example 2: Preparation of an Optical Model

A series of optical models was constructed in accordance with the present invention comprising the following elements:
white diffusing reflective textile substrate;
Nichia 204 side emitting LED, height=0.4 mm;
silicone light guide, layer thickness=1 mm;
white reflective mask over LED, radius=3.5 mm;
black rings for angular selection, inner radius=2.5 mm, outer radius=3.5 mm;
sacrificial layer made from wax, inner radius=3.5 mm, outer radius=8.0 mm.

The following models were constructed based on an LED arranged on a textile substrate and a light guide coating the light emitting element and the substrate:
(a) one LED, no mask, no air gap;
(b) one LED, one mask over the LED, no air gap;
(c) one LED, one mask over the LED, one air gap at interface between substrate and light guide.

The modeling system used was the optical ray trace software which is available from LightTools. The peak illuminance was calculated. In (a) a bright peak was observed at the side of the LED. The peak illuminance was 85×10³ lm/m².

In (b) a shadow was apparent caused by the presence of the mask. The peak illuminance was 21×10³ lm/m².

In (c), it was evident that light was extracted significantly further away from the LED when compared with the other systems in (a) and (b) thus providing a more uniform light output. The peak illuminance was 5×10³ lm/m².

A further experiment was undertaken by modeling the effect of depositing a further light guide of 2 mm of silicone on the first light guide. The addition of the second layer significantly improved the light uniformity.

The results indicated that significant improvements were achieved using the light emitting device in accordance with the present invention.

The person skilled in the art realizes that the present invention is by no means limited to the preferred embodiments described above. On the contrary, many modifications and variations are possible within the scope of the appended claims. For example, the use of top emitting LED(s) may be used in conjunction with or instead of side emitting LEDs.

Additionally, variations to the disclosed embodiments can be understood and effectuated by the skilled person in practicing the claimed invention, from a study of the drawings, the disclosure, and the appended claims. In the claims, the word “comprising” does not exclude other elements or steps, and the indefinite article “a” or “an” does not exclude a plurality. The mere fact that certain features are recited in mutually different dependent claims does not indicate that a combination of these features cannot be used to advantage.

1. A light emitting device comprising:
at least one light emitting element arranged on a first surface of a textile substrate;
a first light guide arranged to coat the at least one light emitting element;
wherein the first light guide has a back surface facing the first surface of the textile substrate and a front surface facing away from the textile substrate;
wherein the back surface of the first light guide, is in direct contact with the first surface of the textile substrate and forms an interface with the first surface of the textile substrate, wherein one or more air gaps are present in the light guide at the interface with the textile substrate for providing a local area of detachment of the first light guide from the textile substrate, and wherein the surface of the one or more air gaps facing the front surface of the first light guide is textured,
and wherein a mask is deposited on the front surface of the first light guide and over the at least one light emitting element.

2. The light emitting device according to claim 1, wherein the at least one light emitting element comprises an LED or a plurality of LEDs.

3. The light emitting device according to claim 1, wherein the at least one light emitting element comprises a side emitting LED or a plurality of side emitting LEDs.

4. The light emitting device according to claim 1, wherein the device further comprises extraction features arranged to extract light through one or both of the front surface and the back surface of the first light guide.

5. The light emitting device according to claim 1, wherein the first light guide is a transparent polymer.

6. The light emitting device according to claim 1, wherein the first light guide is selected from a layer of silicone, polyvinylchloride or polyurethane.

7. The light emitting device according to claim 1, wherein the mask comprises one or more of scattering, reflective and absorbing structures.

8. The light emitting device according to claim 1, wherein a further mask is deposited between the light emitting element and the surface of the textile substrate.

9. The light emitting device according to claim 1, wherein a further light guide is deposited on the first light guide.

10. A method of forming a light emitting device comprising:
arranging at least one light emitting element on a first surface of a textile substrate;
depositing a sacrificial material on said first surface of the textile substrate, wherein the sacrificial material has a textured surface at the surface facing away from the first surface;
coating the at least one light emitting element, the first surface of the textile substrate and the sacrificial material with a first light guide;
removing the sacrificial material for providing a local area of detachment of the first light guide from the textile substrate;
depositing a mask on the light guide and over the at least one light emitting element.

11. The method according to claim 10, wherein the removal of the sacrificial material comprises dissolving the sacrificial material in a solvent or exposing the sacrificial material to heat.

12. The method according to claim 10, wherein the sacrificial material is selected from polyvinylalcohol, guar or beeswax.

13. The method according to claim 10, wherein a second light guide is deposited on the first light guide.

14. An article or device comprising the light emitting device according to claim 1.

15. The article or device according to claim 14, wherein the article or device is selected from a lighting device or a health care device.
